(19) United States
(12) Patent Application Publication
(10) Pub. No.: US 2024/0264574 A1
(43) Pub. Date: Aug. 8, 2024

(54) METHODS, SYSTEMS, AND DEVICES FOR AUTOMATED PERSONALIZED CONTROL OF BUILDING SYSTEMS

(71) Applicant: Research Triangle Institute, Research Triangle Park, NC (US)

(72) Inventors: Lynn Davis, Holly Springs, NC (US); Meghan Sarah Hegarty-Craver, Durham, NC (US); Howard Jerome Walls, Apex, NC (US); Karmann C. Riter, Apex, NC (US)

(21) Appl. No.: 18/433,523

(22) Filed: Feb. 6, 2024

Related U.S. Application Data
(60) Provisional application No. 63/483,424, filed on Feb. 6, 2023.

(51) Int. Cl. G05B 19/042; G16H 40/63

(52) U.S. Cl. CPC G05B 19/042; G16H 40/63; G05B 2219/25011

(57) ABSTRACT

Disclosed herein are methods, systems, and devices for automated personalized control of building systems. According to one embodiment, a method is implemented on a computing system. The method includes receiving wearable device health data associated with a plurality of occupants associated with a building and determining a plurality of settings for a plurality of appliances associated with the building based on the wearable device health data.

### CROSS-REFERENCE TO RELATED APPLICATION

This application claims the benefit of U.S. Provisional Patent Application No. 63/483,424, filed on Feb. 6, 2023, titled “METHODS, SYSTEMS, AND DEVICES FOR AUTOMATED PERSONALIZED CONTROL OF BUILDING SYSTEMS,” the entire contents of which are incorporated herein by reference.

### TECHNICAL FIELD

The present invention relates generally to control of building systems and more specifically automated personalized control of building systems.

### BACKGROUND

Market reports estimate that the global market size for equipment and services for smart buildings was over $70 billion in 2021 and the market is estimated to grow to over $120 billion by 2026. These estimates do not include all of the building automatic systems market, just the segment of the market associated with smart buildings. Government and commercial buildings are expected to account for the majority of this growth. North America is projected to be the largest market at over thirty percent and have over a nine percent compound annual growth rate (CAGR). Asia Pacific is expected to have the highest CAGR at over thirteen percent.

As such, new methods, systems, and devices are needed for better efficiency that also out focus on personalized comfort within building automation systems.

### SUMMARY

This disclosure presents methods, systems, and devices for automated personalized control of building systems. According to one embodiment, a method is implemented on a computing system. The method includes receiving wearable device health data associated with a plurality of occupants associated with a building and determining a plurality of settings for a plurality of appliances associated with the building based on the wearable device health data.

In some embodiments, the method may further include transmitting the plurality of settings to the plurality of appliances. In other embodiments, the method may include transmitting the plurality of settings to an automation system configured to control the plurality of appliances. In some embodiments, the automation system may be a home automation system. In other embodiments, wherein the automation system may be a building automation system.

In some embodiments, the wearable device health data may be obtained from a plurality of wearable monitoring devices associated with the plurality of occupants.

In some embodiments, the plurality of wearable monitoring devices may include at least one wearable physiological monitoring device.

In some embodiments, the plurality of wearable monitoring devices may include at least one wearable fitness tracker device.

In some embodiments, the plurality of wearable monitoring devices may include at least one smart watch.

In some embodiments, the plurality of wearable monitoring devices may include at least one wearable electrocardiogram (ECG) device.

In some embodiments, the plurality of wearable monitoring devices may include at least one blood pressure monitoring device.

In some embodiments, a first portion of the wearable device health data may be captured by the plurality of wearable monitoring devices within one hour of determining the plurality of settings for the plurality of appliances and a second portion of the wearable device health data may be captured by the plurality of wearable monitoring devices more than an hour previous from determining the plurality of settings for the plurality of appliances.

In some embodiments, the wearable device health data may include a plurality of vital signs associated with the plurality of occupants.

In some embodiments, the wearable device health data may include heart rate data associated with the plurality of occupants.

In some embodiments, the wearable device health data may include heart rate variability (HRV) data associated with the plurality of occupants.

In some embodiments, the wearable device health data may include beat-to-beat (RR) interval data associated with the plurality of occupants.

In some embodiments, the wearable device health data may include gait data associated with the plurality of occupants.

In some embodiments, the wearable device health data may include sleep activity data associated with the plurality of occupants.

In some embodiments, the wearable device health data may include movement data associated with the plurality of occupants.

In some embodiments, the wearable device health data may include light exposure data associated with the plurality of occupants.

In some embodiments, the wearable device health data may include ambient temperature exposure data associated with the plurality of occupants.

In some embodiments, the wearable device health data may include ambient humidity exposure data associated with the plurality of occupants.

In some embodiments, the wearable device health data may include electrodermal response data associated with the plurality of occupants.

In some embodiments, the wearable device health data may include respiration data associated with the plurality of occupants.

In some embodiments, the wearable device health data may include oxygen level (SpO2) data associated with the plurality of occupants.

In some embodiments, the wearable device health data may include skin temperature data associated with the plurality of occupants.

In some embodiments, the wearable device health data may include core body temperature (CBT) data associated with the plurality of occupants.

In some embodiments, the wearable device health data may include activity data associated with the plurality of occupants.

In some embodiments, the wearable device health data may include coughing data associated with the plurality of occupants.

In some embodiments, the wearable device health data may include circadian rhythm data associated with the plurality of occupants.

In some embodiments, the plurality of settings may include at least one temperature setting associated with a heating, ventilation, and air conditioning (HVAC) system. In further embodiments, the plurality of settings may include at least one humidity setting associated with the HVAC system. In further embodiments, the plurality of settings may include at least one fan setting associated with the HVAC system.

In some embodiments, the plurality of settings may include at least one brightness setting associated with at least one light fixture.

In some embodiments, the plurality of settings may include at least one hue setting associated with at least one light fixture.

In some embodiments, the plurality of settings may include at least one light direction setting.

In some embodiments, the plurality of settings may include at least one plug load control setting.

In some embodiments, the plurality of settings may include at least one ventilation setting. In further embodiments, the at least one ventilation setting may include a ventilation rate setting. In further embodiments, the at least one ventilation setting may include an airflow direction setting. In further embodiments, the at least one ventilation setting may include an airflow pattern setting. In further embodiments, the at least one ventilation setting may be associated with a natural ventilation system. In further embodiments, the at least one ventilation setting may be associated with a mechanical ventilation system. In still further embodiments, the at least one ventilation setting may be associated with a mixed-mode ventilation system.

In some embodiments, the plurality of settings may include at least one time-based setting.

In some embodiments, the plurality of settings may include at least one motorized blind setting.

In some embodiments, the plurality of settings may include at least one motorized shade setting.

In some embodiments, the plurality of settings may include at least one motorized window setting.

In some embodiments, the plurality of settings may include at least one dimmer setting association with at least one solar tube.

In some embodiments, the plurality of settings may include at least one daylight control setting associated with an automated fixture allowing access to daylight.

In some embodiments, the method may further include receiving sensor data associated with the plurality of appliances and determining the plurality of settings for the plurality of appliances may be further based on the sensor data.

In some embodiments, the plurality of occupants may include a plurality of patients associated with the building. In further embodiments, the plurality of occupants may include a plurality of healthcare workers associated with the building.

In some embodiments, the method may further include receiving medical record health data associated with the plurality of occupants associated with the building and determining the plurality of setting for the plurality of appliances may be further based on the medical record health data. In further embodiments, the medical record health data may be received from at least one medical record database.

In some embodiments, the method may further include receiving occupant entered health data associated with the plurality of occupants associated with the building and determining the plurality of setting for the plurality of appliances may be further based on the occupant entered health data. In further embodiments, the occupant entered health data may be received from a plurality of mobile devices associated with the plurality of occupants associated with the building. In further embodiments, the occupant entered health data may include medical history associated with the plurality of occupants associated with the building. In further embodiments, the occupant entered health data may be received from a plurality surveys completed by the plurality of occupants associated with the building.

In some embodiments, the wearable device health data may be received over a network. In further embodiments, the network may include a wide area network (WAN). In further embodiments, the network may include a local area network (LAN). In further embodiments, the network may include a personal area network (PAN). In further embodiments, the network may include the Internet.

In some embodiments, the method may be executed within a networked computing environment. In further embodiments, the method may be executed on at least one virtualized server within the networked computing environment. In further embodiments, the method may be executed within at least one container within the networked computing environment. In still further embodiments, the at least one container may be a Docker® container.

In some embodiments, the wearable device health data may be received from a plurality of mobile devices associated with the plurality of wearable monitoring devices associated with the plurality of occupants.

In some embodiments, the plurality of mobile devices may include a smart phone, a smart tablet, a smart watch, a laptop, and/or the like.

According to another embodiment, a computing system includes at least one processor and at least one memory electrically coupled with the at least one processor. The computing system is configured for receiving wearable device health data associated with a plurality of occupants associated with a building and determining a plurality of settings for a plurality of appliances associated with the building based on the wearable device health data.

In some embodiments, the computing system may be further configured for transmitting the plurality of settings to the plurality of appliances. In other embodiments computing system may be further configured for transmitting the plurality of settings to an automation system configured to control the plurality of appliances.

According to another embodiment, a non-transitory computer-readable storage medium stores instructions to be implemented on a computing system including at least one processor is disclosed. The instructions when executed by the at least one processor cause the computing system to perform a method. The method includes receiving wearable device health data associated with a plurality of occupants associated with a building and determining a plurality of settings for a plurality of appliances associated with the building based on the wearable device health data.

In some embodiments, the method may further include transmitting the plurality of settings to the plurality of appliances. In other embodiments, the method may include transmitting the plurality of settings to an automation system configured to control the plurality of appliances.

According to another embodiment, a method is implemented on a computing system. The method includes receiving wearable device health data associated with an occupant associated with a building and determining a setting for an appliance associated with the building based on the wearable device health data.

In some embodiments, the wearable device health data may be obtained from a wearable monitoring device associated with the occupant.

In some embodiments, the method may further include transmitting the setting to the appliance. In other embodiments, the method may further include transmitting the setting to an automation system configured to control the appliance.

According to another embodiment, a computing system includes at least one processor and at least one memory electrically coupled with the at least one processor. The computing system is configured for receiving wearable device health data associated with an occupant associated with a building and determining a setting for an appliance associated with the building based on the wearable device health data.

In some embodiments, the computing system may be further configured for transmitting the setting to the appliance. In other embodiments, the computing system may be further configured for transmitting the setting to an automation system configured to control the appliance.

According to another embodiment, a non-transitory computer-readable storage medium stores instructions to be implemented on a computing system including at least one processor is disclosed. The instructions when executed by the at least one processor cause the computing system to perform a method. The method includes receiving wearable device health data associated with an occupant associated with a building and determining a setting for an appliance associated with the building based on the wearable device health data.

In some embodiments, the method may further include transmitting the setting to the appliance. In other embodiments, the method may further include transmitting the setting to an automation system configured to control the appliance.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

FIG. 1 depicts a system diagram illustrating one embodiment of an automated personalized building control system in accordance with embodiments of the present disclosure.

FIG. 2 depicts a block diagram of one embodiment of the wearable monitoring device of FIG. 1 in accordance with embodiments of the present disclosure.

FIG. 3 depicts a system diagram illustrating an automation system configured for managing a plurality of appliances within a building in accordance with embodiments of the present disclosure.

FIG. 4 depicts a block diagram illustrating one embodiment of a mobile device of FIG. 3 in accordance with embodiments of the present disclosure.

FIG. 5 depicts a block diagram illustrating one embodiment of a server of FIG. 3 in accordance with embodiments of the present disclosure.

### DETAILED DESCRIPTION

The following description and figures are illustrative and are not to be construed as limiting. Numerous specific details are described to provide a thorough understanding of the disclosure. However, in certain instances, well-known or conventional details are not described in order to avoid obscuring the description. References to “one embodiment” or “an embodiment” in the present disclosure can be, but not necessarily are, references to the same embodiment and such references mean at least one of the embodiments.

Disclosed herein are methods, systems, and devices for automated personalized control of building systems. These building systems include building automation and control systems for heating, ventilation, and air conditioning (HVAC), lighting, and plug loads. The current solution is mainly manual (e.g., turn on light switch, set a thermostat) and does not change as needs change without further manual intervention. Semi-automated building controls rely on general rules about occupancy such as using these settings during work hours (e.g., 7:00 AM to 6:00 PM) and higher setbacks at off-hours. However, there is no customization to the individual and no automatic response to individual needs.

The present disclosure uses data collected from a wearable monitoring device (e.g., smart watch, smart ring, etc.) to automatically calculate the local settings for lighting, ventilation, and plug load response for at least a section of a building. Areas of the building under local control (and responsive to readings from the wearable monitoring device) may include offices, workspaces, and hallways. Common areas such as entrances and break rooms remain under control of the master system. Areas such as conference rooms that may or may not involve multiple occupants may be under local control (i.e., determined from wearable device health data) for low occupancy and master control (i.e., preset values) once an occupant threshold is exceeded (e.g., three occupants). At a minimum, the wearable monitoring device may function as an occupancy indicator for the master control system. Data collected from the wearable monitoring device may include but is not limited to activity data, heart rate (HR), heart rate variability (HRV), electrodermal response, respiration, oxygen levels (SpO2), skin temperature, core body temperature (CBT), respiration, light exposure, coughing, and circadian rhythm. This wearable device health data is used to calculate various physiological responses of the occupant, and the resulting parameters are provided to a control system that provides settings for at least one building system (e.g., lighting, plug loads, ventilation).

Buildings are one of the largest consumers of energy in the modern world. Three types of building systems (i.e., ventilation, lighting, and plug loads) account for the bulk of electricity usage. These building systems often operate at or near full capacity even when there are no building occupants, which causes the unnecessary energy consumption and contributes to greenhouse gas emissions (GHG). One common solution to this issue is the use of occupancy sensors to inform a control system when a space is occupied. In this way, the building systems are engaged only when needed and either put in standby mode or completely turned off when not needed because no one is present (i.e., the space is unoccupied). However, this approach still uses energy unnecessarily through the standby power of the building systems. In addition, such building systems do not respond to the need of the occupant which may change depending on their physiological state (e.g., level of activity, circadian rhythm, etc.).

A master control system (MCS) adjusts the properties of the lighting control system (LCS) and also the properties of other building systems such as HVAC and plug loads. The LCS may control brightness (i.e., intensity or dimming), correlated color temperature (CCT) via a hue setting, and/or light distribution/direction based upon desired lighting properties of the space. In addition, the MCS can operate controllable outlets and can turn off outlets that are not in use. Plug loads comprise roughly 47% of the electricity consumption in commercial buildings and the fraction of building energy consumption attributable to plug loads is expected to increase as discussed in the National Renewable Energy Laboratory (NREL) publication titled “Assessing and Reducing Plug and Process Loads in Office Buildings” (e.g., https://www.nrel.gov/docs/fy20osti/76994.pdf).

A typical LCS may include a programmable driver (for controlling the light fixtures), and the programmable driver may utilize common communications protocols such as digital multiplex (DMX), digitally addressable lighting interface (DALI), and/or the like. The MCS (and the LCS) can be accessible through a wireless app (e.g., FineTune® by Finelite®) and/or there may be a cable between the MCS and a control pad. The LCS may control lighting both overhead lights (e.g., troffers, panels, downlights, etc.) and lamps connected as plug loads (e.g., task lights, floor lights). Overhead lights are typically operated in a control zone subject to inputs from a master controller such as a LCS, MCS, or a BAS. Task and floor lights are typically controlled more locally (e.g., example in an office or workspace). The lighting system can be fixed CCT or the CCT can be tuned through application of appropriate control signals to the fixture. Both tunable white and tunable color fixtures are within control of the herein disclosed subject matter. For example, tunable white solutions may include one or more of RTI's Next Integrating Classroom Lighting Solutions (NICLS) (e.g., https://www.energy.gov/eere/ssl/rti-international-and-finelite-develop-luminaires-advanced -lighting-classroom).

Tunable color solutions may include one or more of Color Kinetics® systems (e.g., https://www.colorkinetics.com/global).

Tunable lighting fixtures are known to promote healthy circadian rhythms and improved acuity of occupants. Human physiological response to light is discussed in R. J. Lucas et al. (2014) “Measuring and using light in the melanopsin age” Trends in Neurosciences vol. 37 no. 1 p. 1, C. Vetter et al. (2022), “A review of human physiological responses to light: implications for the development of integrative lighting controls” Leukos vol. 18 no. 3 p. 387-414, doi: 10.1080/15502724.2021.1872383, and T. M. Brown et al. (2022) “Recommendations for daytime, evening, and nighttime indoor light exposure to best support physiology, sleep, and wakefulness of health adult” PLOS Biology vol. 20 no. 3 e3001571.

Disclosed as follows is an automated personal building control system that can either be implemented in a building via a building automation system (BAS), locally in a building zone, and/or via a lighting control system (LCS). The automated personal building control system can set the building or zone operational parameters (e.g., ventilation, lighting, plug loads, etc.) to the needs and preferences of the occupant as determined by readings obtained from a wearable monitor device and preferences that are pre-programmed into a look-up table. In instances of conflict of settings by two or more occupants, the global settings may revert to a default value while local settings would still be controlled by individual needs and preferences. The automated personal building control system can work with multiple market sub-sectors. The most likely markets include office buildings and/or healthcare buildings (e.g., hospitals, nursing homes, etc.) but may be used in other building types as well (e.g., stores, factories, warehouses, homes, etc.).

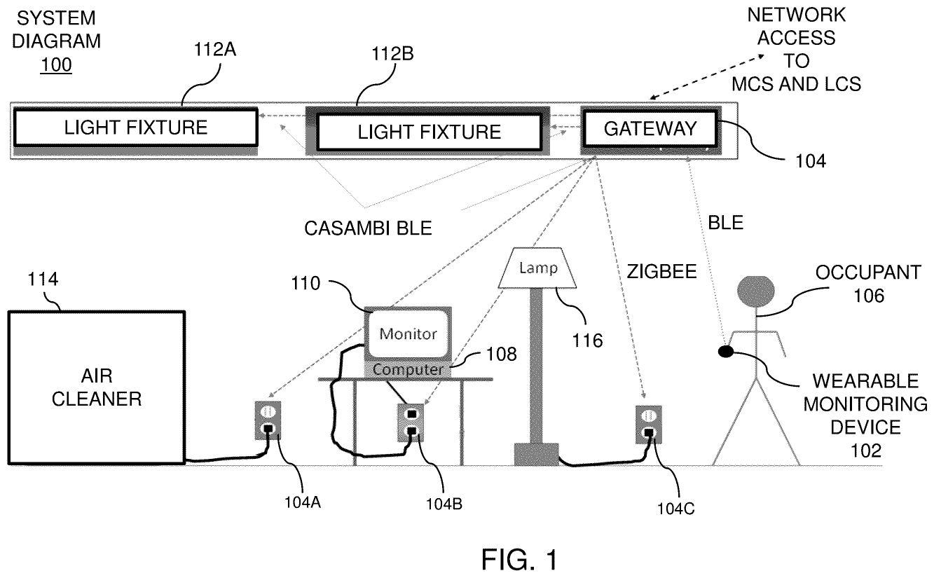

FIG. 1 depicts a system diagram 100 illustrating one embodiment of an automated personalized building control system in accordance with embodiments of the present disclosure. The automated personalized building control system includes a wearable monitoring device 102, one or more controlled receptacles 104A-104C (i.e., smart outlets), a physiological data collection system (PDCS), a master control system (MCS), and a lighting control system (LCS). The PDCS, MSC, and LCS are not shown in FIG. 1 but are networked from another location in the building and/or from a cloud computing facility via a gateway 104.

The wearable monitoring device 102 is worn by an occupant 106 and is capable of measuring physiological parameters. The wearable monitoring device 102 has wireless interface and a unique identification number to allow differentiation between other wearable monitoring devices not shown in FIG. 1. The wearable monitoring device 102 is configured for monitoring a variety of human physiological parameters including one or more of heart rate, SpO2, skin temperature, CBT, sleep state, activities/movement, electrocardiogram (ECG), respiration, light exposure, coughing, and blood/or pressure. The wearable monitor device 102 may also measure environmental factors such as air temperature, light levels, and indoor air quality (IAQ). Examples of such a wearable monitoring device 102 are manufactured by Garmin®, Apple®, Withings®, Corsano®, greenTEG®, and RTI®. Typically, the wearable monitoring device 102 is connected via Bluetooth Low Energy® (BLE) to a mobile device (e.g., a smart phone, a smart tablet, etc.) not shown in FIG. 1. The mobile device that hosts a mobile app for operating the wearable monitor device 102 and recording data. For example, the Wearables Research and Analytics Platform (WRAP) mobile device application may be used (e.g., https://www.rti.org/brochures/wrap % E2% 84% A2-wearables-research-and -analytics-platform-end-end-solution-securely-collecting-and).

The mobile app may record preferences of the occupant 106. In addition, the mobile app may use the measured physiological parameters to calculate other variables such as heart rate variability (HRV), RR intervals, gait analysis, and/or CBT. For example, one or more algorithms developed for U.S. Army. Research Institute of Environmental Medicine (USARIEM) may be used. The PDCS is configured to receive, record, and analyze physiological data (i.e., the wearable device health data) obtained by the wearable monitoring device 102.

The controlled receptacles 104A-104C have electronic relays allowing the MCS to individually switch between ON and OFF states. As shown in FIG. 1, controlled receptacle 104B has one outlet that is always on for a computer 108 and another outlet that is under control of the MCS for a monitor 110 (e.g., a display device). Additionally, the controlled receptacle 104A allows the MCS to control an air cleaner 114 and controlled receptacle 104C allows the MCS to control a lamp 116.

The controllable receptacles 104A-104C may be compliant to at least one version of the ASTM/ASHRAE/IES standard 90.1-2019 and at least one version of the National Electrical Code (NEC) standard. The controllable receptacles 104A-104C may include programmable circuit breakers and wireless Zigbee connectivity via the gateway 104 with the MCS. The controllable receptacles 104A-104C may have two or more outlets. Other wireless communication protocols with the gateway 104 may be used (e.g., Wi-Fi, Z-Wave, Bluetooth®, etc.). The MCS may be used to turn OFF and ON the controllable receptacles 104A-104C and/or to reduce the power delivered (e.g., dimming, etc.). Examples of receptacles where at least one outlet can be controlled include GE® lamp module ZW3103, Lutron® Decora, Lutron® Claro and automatic receptacles such as those described in the article titled “The relationship between lighting and plug load control” in Smart Buildings Technology magazine dated Sep. 20, 2021.

An outlet control switch capability may be located at any point of the power line including built into the receptacle or outlet (as shown with the controllable receptacles 104A-104C of FIG. 1), as a control port between the outlet and the plug load (as occurs with smart switches and dimmers), embedded into the plug load, or as a wireless switch placed between the receptacle and the junction box or electrical panel. In some embodiments, only a fraction of the outlets on a receptacle are controllable from a remote signal from the MCS. For example, it may be advantageous to have certain plug loads such as computers operate continuously while other plug loads (e.g., computer monitors, air cleaners, etc.) may be on controllable receptables (as shown in FIG. 1). In some embodiments, controlled and non-controlled outlets may be contained within the same receptacle and color coded as such.

The MCS is further configured to control the settings of the light fixtures 112A-112B via the LCS using Casambi® BLE. The light settings may include both individual brightness, direction, and hue (e.g., color temperature) settings of the light fixtures 112A-112B. The light settings are determined by the PDCS using the physiological data (i.e., wearable device health data).

The PDCS may include a personal computer, workstation, server, smart tablet, smart phone, cloud resource, edge resource, or other computational device that is capable of collecting, logging, and analyzing physiological data (i.e. wearable device health data) collected by the wearable monitoring device 102. The PDCS can also be attached to a network containing stored data (e.g., a cloud database) such that baseline data may be retrieved when needed for calculations. In this manner, the baseline data may be available to any local PDCS through the cloud. This baseline data including prior history and preferences may be used to set control parameters in new areas of the building.

As discussed, the MCS controls the operation of the LCS and the controllable receptacles 104A-104C. The MCS receives settings from the PDCS as well as any other sensors (e.g., stand-alone occupancy, CO2, daylight harvesting, etc.). In controlling the LCS, the MCS operates like a lighting control system such as described in the article titled “RTI International and Finelite Develop Luminaires for Advanced Lighting in the Classroom” (e.g., https://www.energy.gov/eere/ssl/rti-international-and-finelite-develop-luminaires-advanced-lighting-classroom).

The MCS may implement one or more methods for providing automated personalized control. For example, a control sequence may be initiated when the occupant 106 enters the space to be controlled. The PDCS senses that the wearable monitoring device 102 device is present from the radio emissions from the wearable monitoring device 102 device and interrogates the wearable monitoring device 102 device to determine its identification. This interrogation may be directly between the PDCS and the wearable monitoring device 102 or may involve the mobile device and mobile app as discussed earlier. Wireless signal strength may be a determining factor on whether the mobile device is necessary.

If multiple wireless access points are available (e.g., a large conference room or an open office space), the location of the occupant 106 may be determined by triangulating wireless links to the wearable monitoring device 102.

After a unique identifier (ID) is determined from the wearable monitor device 102, a lookup table of preferred settings may be called by either the MCS or the PDCS. These preferred settings allow the MCS to set the light fixtures 112A-112B and the controllable receptacles 104A-104C to previously entered settings or previously determined settings of the occupant 106.

While the occupant 106 remains in a given area (e.g., control zone, etc.), physiological data (i.e., wearable device health data) collected by the wearable monitoring device 102 may be constantly acquired and analyzed for changes such as activity level, circadian rhythm, health status (e.g., pre-symptomatic), gait direction, and/or the like. If needed, settings may be adjusted for the light fixtures 112A-112B and the controllable receptacles 104A-104C as needed.

For example, if the occupant 106 leaves the space for more than a pre-determined time (e.g., twenty minutes), the light fixtures 112A-112B and the controllable receptacles 104A-104C may be returned to a setting for no occupants. Alternative, controllable receptacles may respond immediately upon an occupant leaving a space in which case the wearable sensor also serves as a vacancy detector since the absence of a BLE sensor from the wearable indicates the space is vacant.

If the occupant 106 exhibits low activity levels, the lighting levels may be adjusted to better suit the lower activity levels. Alternatively, if low activity is detected in an occupant, the lighting levels can be increased to promote alertness.

Overall, the lighting levels (e.g., direction, hue, and/or brightness) may be adjusted to align with a circadian rhythm of the occupant 106. For light within an individual office, hospital room, etc. the lighting levels may be set specifically for the occupant 106. For larger spaces (i.e., hallways, conference rooms, warehouses, etc.), the lighting levels may be preset to some value that changes during the day according to a temporal parameter (e.g., time of day, circadian rhythms, etc.) and by occupancy (which may be determined by the number of unique IDs detected in the space).

Prior physiological data from the wearable monitoring device 102, may provide a baseline that can be accessed by any local PDCS through the cloud. Comparison of current physiological data with historical physiological data may provide insights into physiological changes occurring within the occupant 106. For example, the HRV data, SpO2 data, skin temperature or CBT, or other physiological responses (e.g., coughing, etc.) may be evidence of an infection beginning (i.e., pre-symptomatic detection) or an infection underway. International application published as WO 2023/279082 A1 and titled “SYSTEMS, METHODS, AND DEVICES FOR DETECTING VIRAL RESPIRATORY ILLNESS IN PRESYMPTOMATIC AND ASYMPTOMATIC INFECTED PERSONS” discusses such methods and is incorporated herein in its entirety by reference. Analyzing the current physiological data by the PDCS may promote the initiation of low-regret action such as increasing ventilation rates through the HVAC via the MCS, operating the air cleaner 114 by the controlled receptacle 104A via the MCS, and/or turning on a germicidal ultraviolet (GUV) systems (not shown in FIG. 1).

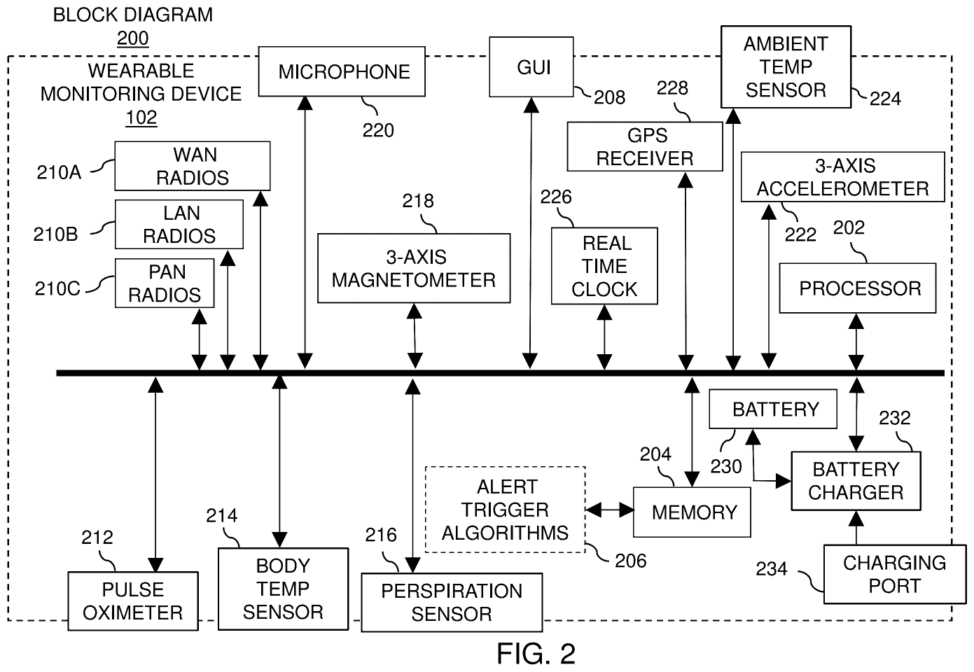

FIG. 2 depicts a block diagram 200 of one embodiment of the wearable monitoring device 102 of FIG. 1 in accordance with embodiments of the present disclosure. The wearable monitoring device 102 is suitable for capturing wearable device health data associated with a given occupant (e.g., the occupant 106 of FIG. 1) of a building and transmitting the wearable device health data to a computing system configured for determining a setting for an appliance associated with the building based on the wearable device health data.

The wearable monitoring device 102 includes a processor 202 and a memory 204. In some embodiments, the memory 204 or a portion of the memory 204 may be integrated with the processor 202. The memory 204 may include a combination of volatile memory and non-volatile memory. In some embodiments the processor 202 and the memory 204 may be embedded in a microcontroller. The processor 202 may be the Snapdragon® 4100 processor, the NXP Kinetix® microcontroller unit (MCU), or the like. The memory 204 is configured for program instructions for capturing the wearable device health data. The program instructions may also include alert trigger algorithms 206 to be used for initiating transmission of the wearable device health data to the computing system. Also (not shown in FIG. 2), a plurality of analog-to-digital converters (ADCs) and digital-to-analog converters (DACs) may be used to interface the various components/sensors to the processor 202.

The wearable monitoring device 102 also includes a graphical user interface (GUI) 308. The GUI 208 may be a touchpad display. The wearable monitoring device 102 also includes wide area network (WAN) radios 210A, local area network (LAN) radios 210B, and personal area network (PAN) radios 210C. The WAN radios 210A may include 2G, 3G, 4G, and/or 5G technologies. The LAN radios 210B may include Wi-Fi technologies such as IEEE 802.11a, 802.11b/g/n, 802.11ac, 802.11.ax or the like circuitry. The PAN radios 210C may include Bluetooth® technologies, IEEE 802.15, and IEEE 802.15.4.

The wearable monitoring device 102 also includes a pulse oximeter 212, a body temperature sensor 214, and a perspiration sensor 216 for obtaining the raw sensor data relating to multiple vital signs of the occupant. The wearable monitoring device 102 also includes a three-axis magnetometer 218, a microphone 220, a three-axis accelerometer 222, and an ambient temperature sensor 224.

The three-axis magnetometer 218 is configured to detect a relative position to gravity of the wearable monitoring device 102. The three-axis accelerometer 222 is configured to detect instantaneous movements on x, y, and z-axis of the wearable monitoring device 202. The microphone 220 may be used to detect background noise that may be indicative of an ongoing activity of the occupant. The ambient temperature sensor 224 may be used to better correlate the occupant's skin temperature from the body temperature sensor 214. Alternatively, a heat flux sensor can be used individually or combined with an ambient or skin temperature sensor to determine core body temperature by measuring heat flux relative to ambient or skin temperature.

The wearable monitoring device 102 also includes a real time clock 226 for time stamping the raw sensor data and a global positioning system (GPS) 228 receiver for determining a location. The wearable monitoring device 102 also includes a battery 230, a battery charger 232, and a charging port 234. The charging port 234 may be a wireless (i.e., contact-less or inductive charging) charging port or a wired (i.e., contact based) charging port.

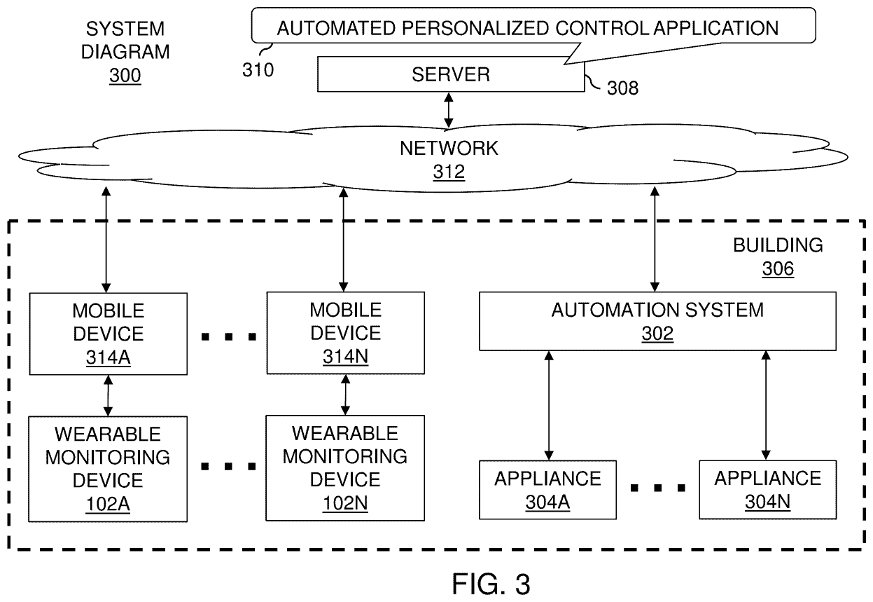

In broader embodiments, FIG. 3 depicts a system diagram 300 illustrating an automation system 302 configured for managing a plurality of appliances 304A-304N within a building 306 in accordance with embodiments of the present disclosure. The automation system 302 is configured to communicate with a server 308 executing an automated personalized control application 310. A network 312 provides connectivity between the automation system 302 and the server 308. The network 312 may include a wide area network (WAN). a local area network (LAN), a personal area network (PAN)., The Internet, cellular (e.g., LTE, 5G, 6G), specialized mobile radios (SMRs), microwave transmitters, and/or the like.

A plurality of wearable monitoring devices 102A-102N are configured to capture wearable device health data from a plurality of occupants of the building 306. A plurality of mobile devices 314A-314N associated with the plurality of occupants are configured to relay the wearable device health data to the server 308 via the network 312.

The automated personalized control application 310 is configured for receiving wearable device health data captured by the plurality of wearable monitoring devices 102A-102N via the network 312 and further configured for determining a plurality of settings for plurality of appliances 304A-304N. The plurality of settings is then transmitted over the network to the automation system 302.

The automated personalized control application 310 and server 310 may be located in a networked computing environment (e.g., a cloud computing environment). The automated personalized control application 310 may also be executed on at least one virtualized server with only a portion running on the server 308. The automated personalized control application 310 may also be executed within a container (e.g., a Docker® container) within the networked computing environment. In further embodiments the server 308 and the automation system 302 may be integrated into the same computing system.

In other embodiments, the automated personalized control application 310 may be configured for transmitting the plurality of settings directly to the plurality of appliances 304A-304N (i.e., either by-passing the automation system 302 or the automation system 302 may not be present at all. The building 306 may include one or more personal residences, a hospital, a skilled nursing facility, offices, a manufacturing facility, a warehouse facility, a store, or the like.

The plurality of wearable monitoring devices 314A-314N may include a fitness tracker device, a smart watch, a smart ring, an ECG device, a blood pressure monitoring device, and/or the like. A first portion of the wearable device health data may be captured by the plurality of wearable monitoring devices 314A-314N within one hour of determining the plurality of settings for the plurality of appliances 304A-304N. A second portion of the wearable device health data may be captured by the plurality of wearable monitoring devices 314A-314N more than an hour previous from determining the plurality of settings for the plurality of appliances 304A-304N.

The wearable device health data may include a plurality of vital signs associated with the plurality of occupants. Additionally, the wearable device health data may include heart rate data, HRV data, RR interval data, gait data, sleep activity data, body movement data, light exposure data, ambient temperature exposure data, ambient humidity exposure data, electrodermal response data, respiration data, SpO2 data, skin temperature data, CBT data, activity data, coughing data, circadian rhythm data, and/or the like associated with the plurality of occupants.

The plurality of settings may include temperature setting data, humidity setting data, fan setting data, damper positions, and/or like associated with an HVAC system (i.e., an appliance 304).

The plurality of settings may also include brightness setting data, hue setting data, and/or light direction setting data associated with a light fixture (i.e., an appliance 304).

The plurality of settings may further include plug load control data, wherein one or more of the plurality of appliances 304A-304N are electrically coupled with controlled receptacles (not shown in FIG. 3).

The plurality of settings may include ventilation setting data. The ventilation setting data may include a ventilation rate setting, an airflow direction setting, an airflow pattern setting, indoor air quality, indoor particulate levels (e.g., PM2.5 levels), indoor volatile organic carbon (VOC) levels, indoor radon levels, and/or the like. The ventilation setting may be associated with a natural ventilation system, a mechanical ventilation system, a mixed-mode ventilation system, and/or the like (i.e., one or more of the plurality of appliances 304A-304N).

The plurality of settings may also include a time-based setting for one or more of the plurality of appliances 304A-304N.

The plurality of settings may include motorized blind setting data. motorized shade setting, motorized window setting, and or the like. The plurality of settings may include daylight control setting data associated with an automated fixture allowing access to daylight (e.g., a solar tube).

The automated personalized control application 310 may be further configured for receiving sensor data associated with the plurality of appliances 304A-304N and determining the plurality of settings for the plurality of appliances 304A-304N may be further based on the sensor data.

The plurality of occupants may include a plurality of patients and or a plurality of healthcare workers associated with the building 306. The automated personalized control application 310 may be further configured for receiving medical record health data associated with the plurality of occupants and determining the plurality of settings for the plurality of appliances 304A-304N may be further based on the medical record health data. The medical record health data may be received from a medical record database over the network 312.

The automated personalized control application 310 may be further configured for receiving occupant entered health data associated with the plurality of occupants and determining the plurality of settings for the plurality of appliances 304A-304N may be further based on the occupant entered health data. The occupant entered health data may be received from the plurality of mobile devices 314A-314N. The occupant entered health data may include medical history associated with the plurality of occupants. The occupant entered health data may be received from a plurality of surveys completed by the plurality of occupants . . .

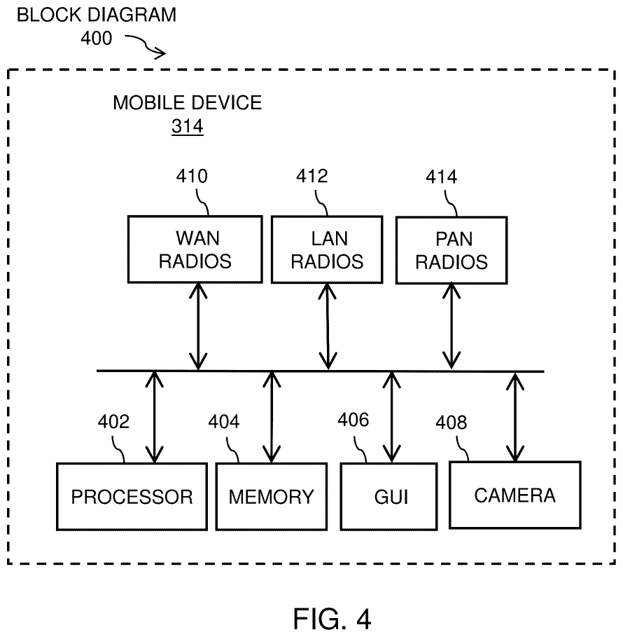

FIG. 4 depicts a block diagram 400 illustrating one embodiment of the mobile device 314 of FIG. 3 in accordance with embodiments of the present disclosure. The mobile device 314 may be a smart phone, a smart tablet, a smart watch, a laptop, or the like. The mobile device 314 may include at least a processor 402, a memory 404, a graphical user interface (GUI) 406, a camera 408, WAN radios 410, LAN radios 412, and PAN radios 414. In some embodiments, the mobile device 314 may be an iPhone® or an iPad®, using iOS® as an operating system (OS). In other embodiments, the mobile device 124 may be a mobile terminal including Android® OS or the like.

In some embodiments, the processor 402 may be a mobile processor such as the Qualcomm® Snapdragon™ mobile processor. The memory 404 may include a combination of volatile memory (e.g., random access memory) and non-volatile memory (e.g., flash memory). The memory 404 may be partially integrated with the processor 402. The GUI 406 may be a touchpad display. The WAN radios 410 may include 2G, 3G, 4G, and/or 5G technologies. The LAN radios 412 may include Wi-Fi technologies such as IEEE 802.11a, 802.11b/g/n, 802.11ac, 802.11.ax and/or the like circuitry. The PAN radios 414 may include Bluetooth® technologies, IEEE 802.15, and 802.15.4.

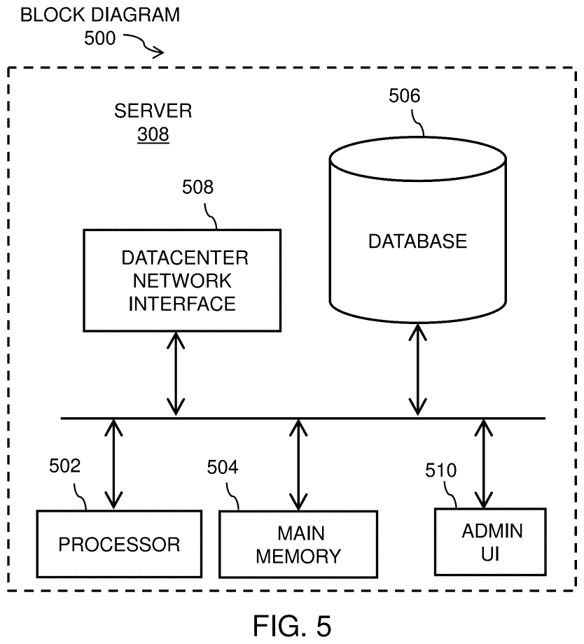

FIG. 5 depicts a block diagram 500 illustrating one embodiment of the server 308 of FIG. 3 in accordance with embodiments of the present disclosure. The server 308 includes at least one processor 504, a main memory 504, a storage memory (e.g., database) 506, a datacenter network interface 508, and an administration user interface (UI) 510. The server 308 may be configured to host at least a portion of a virtual server (e.g., an Ubuntu® server or the like). In some embodiments, the virtual server may be distributed over a plurality of hardware servers (like the server 308) using hypervisor technology.

The processor 502 may be a multi-core server class processor suitable for hardware virtualization. The processor may support at least a 64-bit architecture and a single instruction multiple data (SIMD) instruction set. The main memory 504 may include a combination of volatile memory (e.g., random access memory) and non-volatile memory (e.g., flash memory). The database 506 may include one or more hard drives.

The datacenter network interface 508 may provide one or more high-speed communication ports to data center switches, routers, and/or network storage appliances within a cloud computing environment. The datacenter network interface 510 may include high-speed optical Ethernet, InfiniBand (IB), Internet Small Computer System Interface (iSCSI), and/or Fibre Channel interfaces. The administration UI 510 may support local and/or remote configuration of the server 308 by a datacenter administrator.

The server 308 may be implemented within a cloud computing environment such as the Microsoft Azure®, the Amazon Web Services® (AWS), or the like cloud computing data center environments. The server 308 may also be configured to be hosted within a virtual container. For example, the virtual container may be the Docker® virtual container or the like. In some implementations, the virtual container may be distributed over a plurality of hardware servers using hypervisor technology.

In some embodiment, the server 308 may be a computing device implemented within the building 306. For example, the server 308 may be personal computer (PC), a workstation, an edge computer, and/or the like.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method implemented on a computing system, the method comprising:
receiving sensor data associated with a plurality of appliances associated with a building;
receiving wearable device health data including vital sign data associated with a plurality of occupants associated with the building;
determining a plurality of settings for the plurality of appliances based on the wearable device health data and the sensor data; and
transmitting the plurality of settings to an automation system configured to control the plurality of appliances.

2. The method of claim 1, wherein:
a first portion of the wearable device health data was captured by a plurality of wearable monitoring devices associated with the plurality of occupants within one hour of determining the plurality of settings for the plurality of appliances associated with the building based on the wearable device health data; and
a second portion of the wearable device health data was captured by the plurality of wearable monitoring devices more than an hour previous from determining the plurality of settings for the plurality of appliances associated with the building based on the wearable device health data.

3. The method of claim 1, wherein the wearable device health data includes heart rate data associated with the plurality of occupants.

4. The method of claim 1, wherein the wearable device health data includes heart rate variability data associated with the plurality of occupants.

5. The method of claim 1, wherein the wearable device health data includes beat-to-beat (RR) interval data associated with the plurality of occupants.

6. The method of claim 1, wherein the wearable device health data includes sleep activity data associated with the plurality of occupants.

7. The method of claim 1, wherein the wearable device health data includes movement data associated with the plurality of occupants.

8. The method of claim 1, wherein the wearable device health data includes light exposure data associated with the plurality of occupants.

9. The method of claim 1, wherein the wearable device health data includes ambient temperature exposure data associated with the plurality of occupants.

10. The method of claim 1, wherein the wearable device health data includes electrodermal response data associated with the plurality of occupants.

11. The method of claim 1, wherein the wearable device health data includes oxygen level (SpO2) data, and skin temperature data.

12. The method of claim 1, wherein the wearable device health data further includes respiration data.

13. The method of claim 1, wherein the wearable device health data includes activity data associated with the plurality of occupants.

14. The method of claim 1, wherein the wearable device health data includes coughing data associated with the plurality of occupants.

15. The method of claim 1, wherein the wearable device health data includes circadian rhythm data associated with the plurality of occupants.

16. The method of claim 1, wherein the plurality of settings includes:
at least one brightness setting associated with at least one light fixture;
at least one plug load control setting; and
at least one ventilation setting.

17. The method of claim 1 further comprising receiving medical record health data from at least one medical record database associated with the plurality of occupants associated with the building and determining the plurality of setting for the plurality of appliances is further based on the medical record health data.

18. The method of claim 1 further comprising receiving occupant entered health data associated with the plurality of occupants associated with the building and determining the plurality of setting for the plurality of appliances is further based on the occupant entered health data.

19. A computing system comprising;
at least one processor; and
at least one memory electrically coupled with the at least one processor and storing programming instructions, wherein the computing system is configured for:
receiving sensor data associated with a plurality of appliances associated with a building;
receiving wearable device health data including vital sign data associated with a plurality of occupants associated with the building;
determining a plurality of settings for the plurality of appliances based on the wearable device health data and the sensor data; and
transmitting the plurality of settings to an automation system configured to control the plurality of appliances.

20. A non-transitory computer-readable storage medium, the non-transitory computer-readable storage medium storing instructions to be implemented on a computing system including at least one processor, the instructions when executed by the at least one processor cause the computing system to perform a method, the method comprising:
receiving sensor data associated with a plurality of appliances associated with a building;
receiving wearable device health data including vital sign data associated with a plurality of occupants associated with the building;
determining a plurality of settings for the plurality of appliances based on the wearable device health data and the sensor data; and
transmitting the plurality of settings to an automation system configured to control the plurality of appliances.
